(19) United States
(12) Patent Application Publication
(10) Pub. No.: US 2006/0231115 A1
(43) Pub. Date: Oct. 19, 2006

(54) Support stand for a hair coloring tool

(76) Inventor: Helen McNamara, Waco, TX (US)

Correspondence Address: DOCKET CLERK, P.O. DRAWER 800889, DALLAS, TX, 75380, US

(21) Appl. No.: 11/108,161

(22) Filed: Apr. 15, 2005

(51) Int. Cl. A45D 19/18; A61K 7/13; A61K 7/135

(52) U.S. Cl. 132/208; 132/270

(57) ABSTRACT

A support stand for a hair coloring tool is disclosed that is particularly effective and efficient in supporting a hair coloring tool of the type used for performing foil highlighting and hair coloring. An advantageous embodiment of the hair coloring tool support stand comprises a bowl for containing a hair coloring liquid and a receptacle formed within a wall of the bowl for holding the hair coloring tool. The support stand also comprises a hair coloring tool support structure within the bowl of the support stand. The user may place the hair coloring tool on the hair coloring tool support structure within the bowl. The user may also place the hair coloring tool within the receptacle when the hair coloring tool is not in use.

### FIELD OF THE INVENTION

The present invention relates generally to cosmetic and beauty supplies. More particularly, the present invention relates to an improved support stand for a hair coloring tool of a type used to facilitate foil highlighting of hair.

### BACKGROUND OF THE INVENTION

There are several primary objectives in applying coloring agents to hair. For example, the coloring agent (typically in the form of a liquid, paste or gel) must be applied in a manner that creates a natural appearance. In other words, the application should have an appearance that resembles natural hair color, rather than the appearance of manual coloring. One way to enhance the natural appearance of hair coloring is to avoid lines that clearly demarcate colored hair from uncolored hair. It is also important that the coloring application be quick and inexpensive. Accordingly, the desired natural appearance should not be attained at the expense of time and money. However, conventional hair coloring tools have proven to be ineffective in applying coloring agents in a manner that provides the desired natural appearance, without sacrificing time and expense.

One traditional type of hair coloring technique referred to as “foil highlighting” involves coloring of only portions of the hair. The highlighted or colored hair is typically separated from the rest of the hair by scaling out successive tufts of hair, and a coloring preparation is then applied to the tufts. Strips of metal foil are used to separate the treated tufts from the rest of the hair so as to avoid the highlighting preparation from coming into contact with the rest of the hair during the time that the highlighting preparation requires to take effect. After the highlighting preparation has taken effect the strips of metal foil are removed and the highlighting preparation rinsed off.

The conventional manner of carrying out foil highlighting is as follows. First, a section of hair is scaled out and held between the first and middle fingers of the hair stylist. The thin cylindrical tail of a metal tail comb (“rat tail comb”) is then woven through the section and lifted, thereby separating tufts of hair from the section. The remaining hair of the section is allowed to fall back onto the head of the person whose hair is being done. A strip of metal foil is now inserted underneath the separated tufts, with the foil being firmly advanced and pressed close to the scalp by use of the metal tail of the comb, whereupon the tufts are then released so that they fall back onto the foil. The tail comb is then set down, and a highlighting brush is picked up. A highlighting preparation is then applied to the separated tufts with the brush, using the head of the person whose hair is being done as a support.

The conventional technique is slow and cumbersome, because the tail comb must be put down and the highlighting brush picked up, and then reversed, in alternating use for each tuft. The comb section of the metal tail comb is not used frequently in the foil highlighting process, so the process is significantly hampered and encumbered by the provision of two separate tools for foiling and color application.

One type of hair coloring tool that is particularly useful in carrying out a foil highlighting process comprises an elongated body having a first end and a second end. The first end comprises a thin metal rod for foil highlighting. The second end comprises a highlighting brush. The user of such a hair coloring tool can alternately use each of the two ends of the tool.

It would be convenient if the user of such a hair coloring tool had a support stand that was capable of properly and effectively supporting either end of the tool. There is therefore a need in the art for an improved support stand that is capable of holding and supporting such a hair coloring tool.

### SUMMARY OF THE INVENTION

To address the above-discussed deficiencies of the prior art, it is a primary object of the present invention to provide a support stand for effectively and efficiently holding and supporting a hair coloring tool.

In one advantageous embodiment of the present invention, the support stand of the present invention comprises a bowl for containing a hair coloring liquid and a receptacle formed within a wall of the bowl for holding the hair coloring tool. The support stand also comprises a hair coloring tool support structure within the bowl of the support stand. The user may place the hair coloring tool on the hair coloring tool support structure within the bowl. The user may also place the hair coloring tool within the receptacle when the hair coloring tool is not in use. Placing the hair coloring tool within the receptacle also keeps the brush of the hair coloring tool clean.

In one advantageous embodiment of the invention the hair coloring tool support structure within the bowl comprises a stair structure that extends from the bottom of the bowl to the top of the bowl. When the user desires to place the hair coloring tool within the bowl the user places the hair coloring tool on the stair structure. In another advantageous embodiment of the invention the hair coloring tool support structure comprises a ladder structure that extends from the bottom of the bowl to the top of the bowl.

It is an object of the present invention to provide a hair coloring tool support stand that is effective and efficient in holding and supporting a hair coloring tool.

It is also an object of the present invention to provide a hair coloring tool support stand that comprises a bowl for holding hair coloring liquid and a receptacle within in a wall of the bowl for holding a hair coloring tool.

It is yet another object of the present invention to provide a hair coloring tool support stand that has a hair coloring tool support structure within the bowl of the hair coloring tool support stand for supporting a hair coloring tool.

The foregoing has outlined rather broadly the features and technical advantages of the present invention so that those skilled in the art may better understand the detailed description of the invention that follows. Additional features and advantages of the invention will be described hereinafter that form the subject of the claims of the invention. Those skilled in the art should appreciate that they may readily use the conception and the specific embodiment disclosed as a basis for modifying or designing other structures for carrying out the same purposes of the present invention. Those skilled in the art should also realize that such equivalent constructions do not depart from the spirit and scope of the invention in its broadest form.

Before undertaking the Detailed Description of the Invention below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. Definitions for certain words and phrases are provided throughout this patent document, those of ordinary skill in the art should understand that in many, if not most instances, such definitions apply to prior uses, as well as future uses, of such defined words and phrases.

### BRIEF DESCRIPTION OF THE DRAWINGS

For a more complete understanding of the present invention and its advantages, reference is now made to the following description taken in conjunction with the accompanying drawings, in which like reference numerals represent like parts:

FIG. 1 illustrates a plan view of an advantageous embodiment of a hair coloring tool of a type for use with the support stand of the present invention;

FIG. 2 illustrates a side elevation of the hair coloring tool shown in FIG. 1;

FIG. 3 illustrates a perspective side view of an advantageous embodiment of a hair coloring tool support stand constructed in accordance with the principles of the present invention;

FIG. 4 illustrates a perspective end view of the hair coloring tool support stand shown in FIG. 3.

FIG. 5 illustrates a first perspective top view of the hair coloring tool support stand shown in FIG. 3;

FIG. 6 illustrates a second perspective top view of the hair coloring tool support stand shown in FIG. 3;

FIG. 7 illustrates a schematic cross sectional view of the hair coloring tool support stand taken along the line A-A shown in FIG. 5;

FIG. 8 illustrates an exemplary placement of a hair coloring tool within a bowl portion of the hair coloring tool support stand shown in FIG. 7;

FIG. 9 illustrates an exemplary placement of a hair coloring tool within a receptacle portion of the hair coloring tool support stand shown in FIG. 7;

FIG. 10 illustrates an exemplary hair coloring tool support structure having the form of a ladder structure;

FIG. 11 illustrates a perspective top view of another advantageous embodiment of a hair coloring tool support stand of the present invention having a hair coloring tool support structure that does not extend down to the bottom of the hair coloring tool support stand;

FIG. 12 illustrates a perspective side view of another advantageous embodiment a hair coloring tool support stand of the present invention having a foot portion that is located beneath a receptacle of the hair coloring support stand; and

FIG. 13 illustrates a perspective side view of another advantageous embodiment of the hair coloring tool support stand of the present invention having a base portion beneath a bowl of the hair coloring support stand.

### DETAILED DESCRIPTION OF THE INVENTION

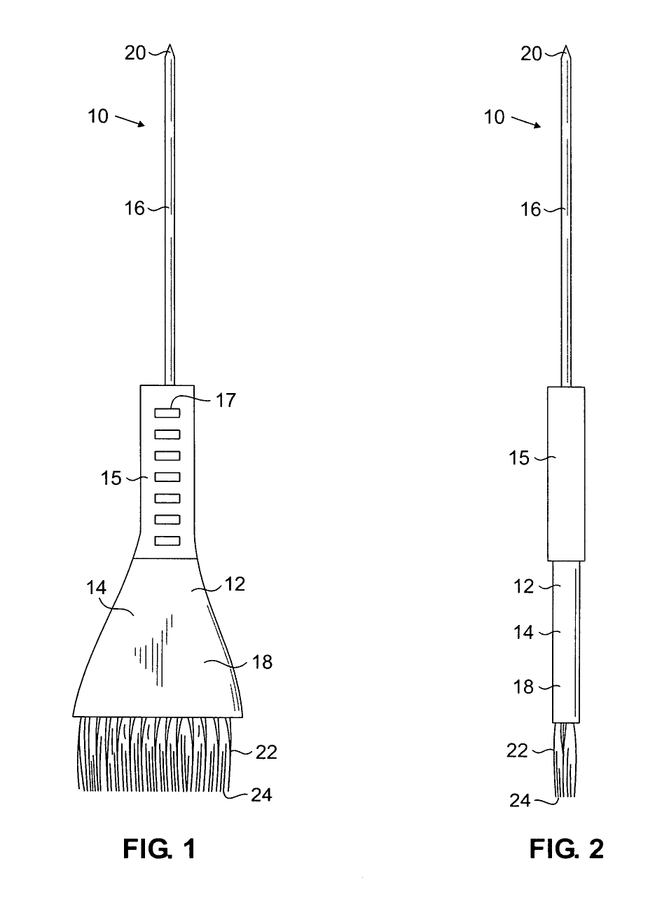

FIGS. 1 through 13 and the various embodiments used to describe the principles of the present invention in this patent document are by way of illustration only and should not be construed in any way to limit the scope of the invention. Those skilled in the art will understand that the principles of the present invention may be implemented in any type of suitably arranged hair coloring tool support stand.

In order to better understand the structure and function of the hair coloring tool support stand of the present invention, the structure and function of an exemplary hair coloring tool will first be described. Turning initially to FIG. 1, hair coloring tool 10 comprises an elongated body 12 having a first end 16 and a second end 14. The first end 16 comprises a thin metal rod that will be used for purposes that will subsequently be described.

Hair coloring tool 10 also comprises a gripping handle 15 that is located between the first end 16 and the second end 14 of elongated body 12. As shown in FIG. 2, the thickness of the gripping handle 15 is thicker than the thickness of the second end 14 of hair coloring tool 10. This permits a user of hair coloring tool 10 to obtain a firm grip on the central portion of hair coloring 10. As the user alternately uses the opposite ends of the hair coloring tool 10 the user continuously rotates the hair coloring tool 10 about an axis that is perpendicular to the longitudinal axis of the hair coloring tool 10. The presence of the gripping handle 15 facilitates the rotation of hair coloring tool 10 by the user.

Gripping handle 15 is also formed having portions that form a plurality of gripping ridges 17 in the surface of gripping handle 15. The gripping ridges 17 provide additional gripping surfaces for the user to contact when gripping the gripping handle 15. The continuous alternate rotation of hair coloring tool 10 by the user is facilitated by the use of the gripping handle 15 and its gripping ridges 17.

The body 12 is relatively larger adjacent the opposite second end 14 and defines a substantially flat head 18. It will also be appreciated that the body 12 presents a substantially central longitudinal axis extending from the first end 16 to the opposite second end 14.

The thin metal rod of the first end 16 has a pointed terminus 20, and preferably a length in the range from approximately three and three fourths inches (3.75 inches) to approximately five and one half inches (5.50 inches). The pointed terminus 20 facilitates the separation of individual tufts of hair. The diameter of the thin metal rod of the first end 16 is substantially constant along its length, and is preferably about one sixteenth of an inch (0.0625 inch) . Steel is the preferred material for the thin metal rod of the first end 16.

The second end 14 of body 12 is formed of any suitable material, such as injection molded plastic, wood, or metal. When a wood material is used, the body 12 may be stained or painted. On the other hand, when the body 12 is formed of a plastic material, a pigment may be added prior to molding for providing the desired coloring. It is noted that the flat head 18 is well suited for indicia, such as a trademark or advertising.

Projecting from the second end 14 of the body 12 is a plurality of flexible bristles 22 arranged in a single row of separate tufts spaced across the head 18. As shown in FIG. 1, the bristles 22 cooperatively define an application brush 24 spaced from the head 18. Each illustrated tuft of bristles has a proximate end (not shown) fixed by suitable means (e.g., glue) within a cylindrical opening (also not shown) extending into the head 18. It is also noted that the preferred tufts have generally the same overall length.

In operation, the user grasps hair coloring tool 10 by the gripping handle 35 while using either the application brush 24 defined by the bristles 22 or the thin metal rod of the first end 16. Function can be quickly alternated between foiling with the thin metal rod of the first end 16 and color application with the application brush 24 of the second end 14, without having to set down and pick up separate tools. In these respects, hair coloring tool 10 is particularly useful in efficiently and effectively applying hair coloring agents to the hair and facilitating foil highlighting.

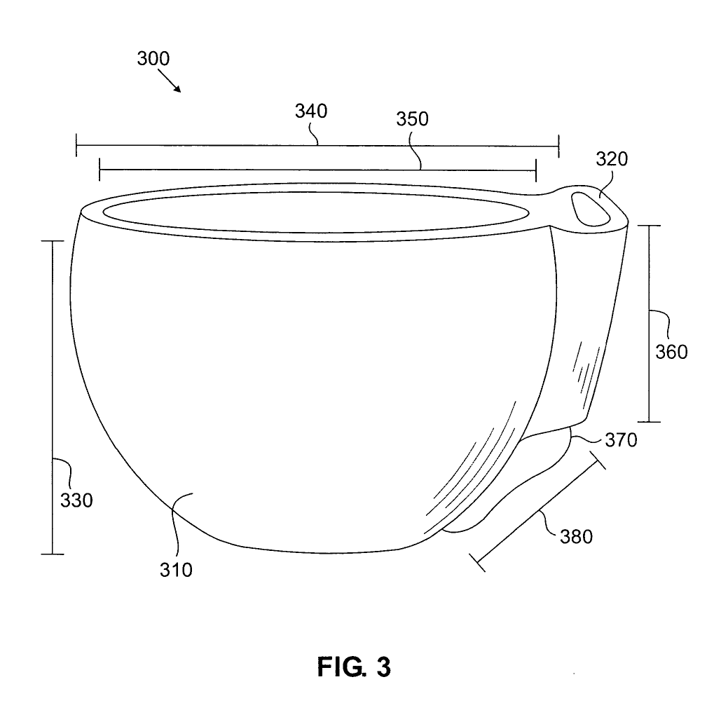

FIG. 3 illustrates a perspective side view of an advantageous embodiment of a hair coloring tool support stand 300 constructed in accordance with the principles of the present invention. Hair coloring tool support stand 300 comprises portions that form a bowl 310 and a receptacle 320. Bowl 310 contains a hair coloring liquid (e.g., hair dye). In operation, the user of hair coloring tool 10 dips the application brush 24 of hair coloring tool 10 within the hair coloring liquid that is within bowl 310. Whenever the user requires additional hair coloring liquid on the application brush 24, the user dips the application brush 24 of hair coloring tool 10 in the hair coloring liquid within bowl 310.

In one exemplary embodiment of hair coloring tool support stand 300 the distance from the bottom of bowl 310 to a maximum fill line for the hair coloring liquid is approximately two and five eighths inches (2.625 inches) . This dimension is shown in FIG. 3 as dimension 330. In this exemplary embodiment of hair coloring tool support stand 300 the external diameter of the top of bowl 310 is approximately four inches (4.0 inches) and the internal diameter of the top of bowl 310 is approximately three and five eighths inches (3.625 inches). These dimensions are respectively shown in FIG. 3 as dimension 340 and dimension 350.

The function of the receptacle 320 is to hold and to support hair coloring tool 10. Whenever the user ceases using hair coloring tool 10, the user places the application brush 24 of hair coloring tool 10 within in receptacle 320. The width of receptacle 320 is sufficient to receive the hair coloring tool 10. When the user releases his or her grip on the hair coloring tool 10, the hair coloring 10 drops into receptacle 320 and the application brush 24 of hair coloring tool 10 comes to rest on the bottom of receptacle 320. Receptacle 320 is sufficiently deep so that the hair coloring tool 10 remains vertically supported within receptacle 320.

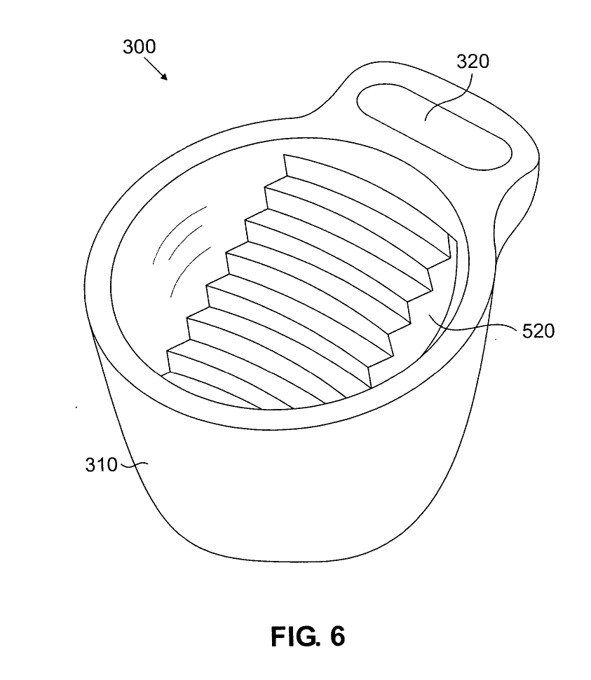

In one exemplary embodiment of hair coloring tool support stand 300 the external distance from the top of receptacle 320 to the bottom of receptacle 320 is approximately one and three eighths inches (1.375 inches). This dimension is shown in FIG. 3 as dimension 360. In this exemplary embodiment of hair coloring tool support stand 300 the internal depth of receptacle 320 is approximately one and two eights inches (1.250 inches). This dimension is not shown in FIG. 3. In this exemplary embodiment of hair coloring tool support stand 300 the width of receptacle 320 is approximately two and five eights inches (2.625 inches). This dimension is shown in FIG. 5 as dimension 510.

Beneath the bottom of receptacle 320 is a foot portion 370 of receptacle 320. The foot portion 370 of receptacle 320 provides structural support for the bottom of receptacle 320 and resists forces that would tend to separate the receptacle 320 from the bowl 310. In this exemplary embodiment of hair coloring tool support stand 300 the height of foot portion 370 of receptacle 320 is approximately one and two eights inches (2.250 inches) . This dimension is shown in FIG. 3 as dimension 380.

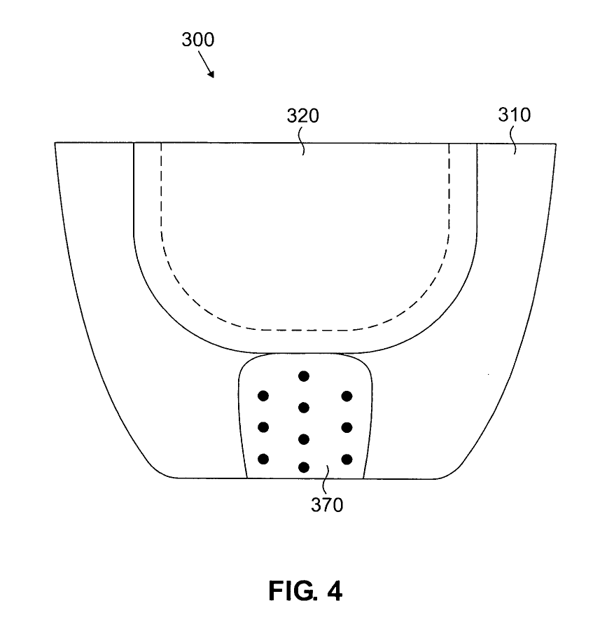

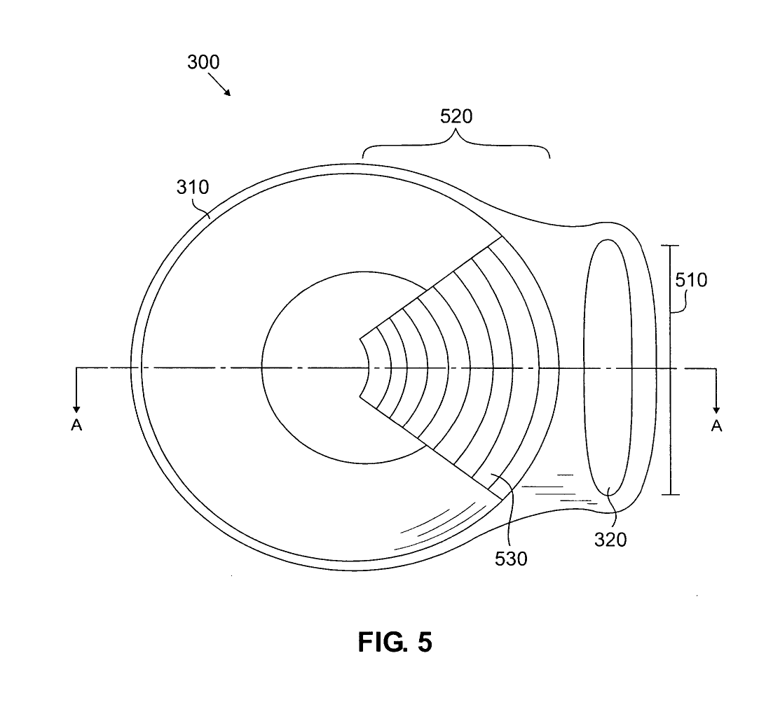

FIG. 4 illustrates a perspective end view of the hair coloring tool support stand 300. FIG. 4 illustrates a direct end view of receptacle 320 and the foot portion 370 of receptacle 320. The dotted line within receptacle 320 represents the interior dimensions of receptacle 320.

FIG. 5 illustrates a perspective top view of the hair coloring tool support stand 300. As previously mentioned, the width of receptacle 320 is shown in FIG. 5 as dimension 510. Also shown in FIG. 5 is a plan view of a hair coloring tool support structure 520 formed adjacent to a wall of bowl 310 that is adjacent to receptacle 320. In the advantageous embodiment shown in FIG. 5 the hair coloring tool support structure 520 comprises a stair structure 520. Stair structure 520 comprises a plurality of steps 530 that extend in stairstep fashion from the bottom of bowl 310 to the top of bowl 310. In one advantageous embodiment each of the plurality of steps 530 is a curved step having a center at the central axis of bowl 310. In one advantageous embodiment the stair structure 520 has the general shape of a circular sector having a center at the central axis of bowl 310 and having as boundaries radii from the central axis of bowl 310 to the edges of the wall of bowl 310 that are adjacent to receptacle 320.

It is understood that the concentric structure of stair structure 520 that has been described is merely one example of the geometry of stair structure 520. Stair structures 520 of other geometric types (e.g., with parallel, non curved steps 530) may also be fashioned and used within bowl 310.

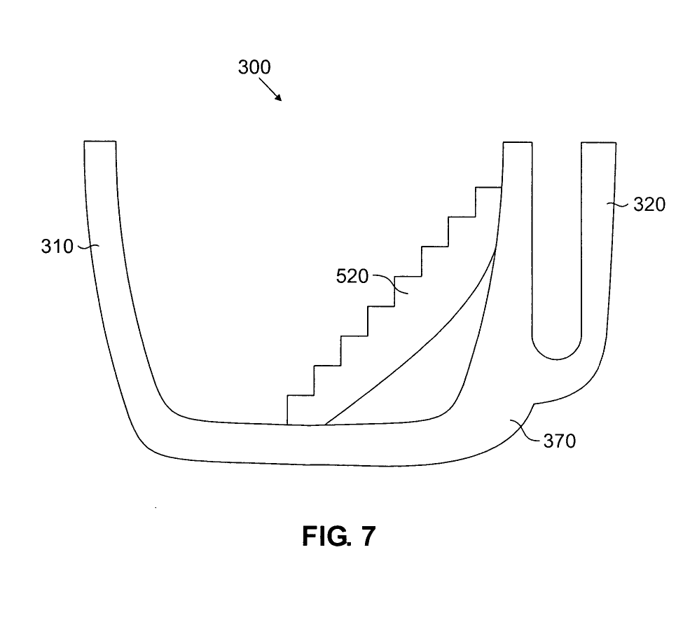

FIG. 6 illustrates a perspective view of hair coloring tool support stand 300. FIG. 6 shows the location of stair structure 520 within bowl 310 of support stand 300 with respect to the location of receptacle 320. FIG. 7 illustrates a schematic cross sectional view of the hair coloring tool support stand 300 taken along the line A-A shown in FIG. 5.

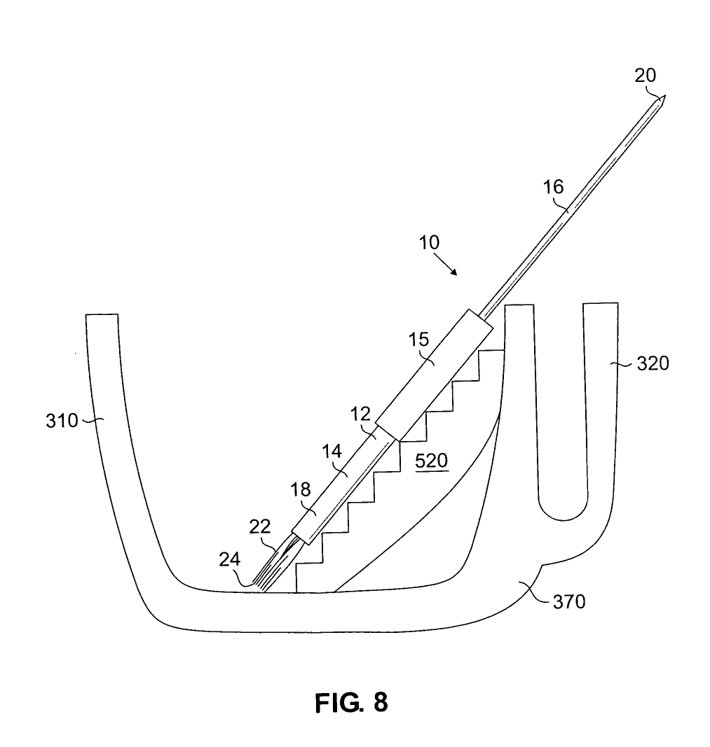

FIG. 8 illustrates an exemplary placement of a hair coloring tool 300 within bowl 310 of the hair coloring tool support stand 300 shown in FIG. 7. When hair coloring tool 10 is placed within bowl 310 then the side of hair coloring tool 10 rests upon the stair structure 520. In this manner the stair structure 520 provides support for hair coloring tool 10.

The steps 530 of stair structure 520 may also be used to remove excess hair coloring liquid from hair coloring tool 10. When the user dips the application brush 24 of hair coloring tool 10 into the hair coloring liquid, the user can remove excess portions of the hair coloring liquid by moving the application brush 24 against one or more steps 530 that are located above the surface of the hair coloring liquid in bowl 310. Because stair structure 520 extends from the bottom of bowl 310 to the top of bowl 310 there will always be some steps 530 that are located above the surface of the hair coloring liquid in bowl 310. The excess hair coloring liquid that falls on the steps 530 flows back down into the reservoir of hair coloring liquid in the bottom of bowl 310.

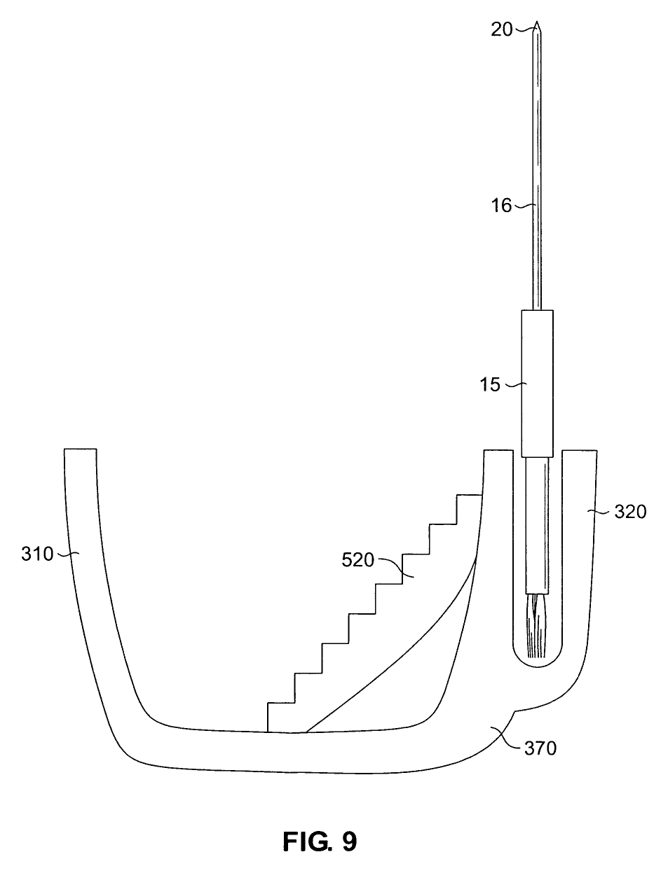

FIG. 9 illustrates an exemplary placement of hair coloring tool 10 within receptacle 320 of the hair coloring tool support stand 300 shown in FIG. 7. As shown in FIG. 9, the application brush 24 of hair coloring tool 10 rests against the bottom of receptacle 320.

The hair coloring tool support stand 300 provides a very useful and efficient support stand for hair coloring tool 10. The user has the convenience of placing hair coloring tool 10 on the stair structure 520 in bowl 310 (within the hair coloring liquid) or within receptacle 320 (away from the hair coloring liquid). This feature allows the user to better control the amount of hair coloring liquid that is present on hair coloring tool 10 during a hair coloring process.

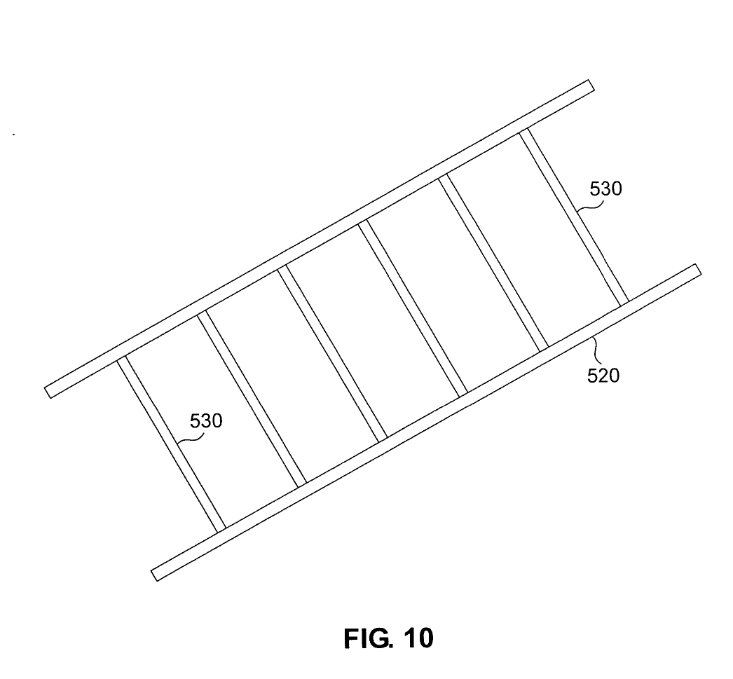

It is understood that the hair coloring tool support structure 520 that has been described as a stair structure 520 is merely one example of a possible geometry for hair coloring tool support structure 520. Other types of hair coloring tool support structures 520 may also be fashioned and used within bowl 310. For example, hair coloring tool support structure 520 may comprise a ladder structure 520 of the type shown in FIG. 10. Ladder structure 520 comprises a plurality of rungs 530 that extend in ladder fashion from the bottom of bowl 310 to the top of bowl 310. In an alternate embodiment of the invention the plurality of rungs 530 of ladder structure 520 do not extend all the way down to the bottom of bowl 310. From this example, it is understood that other types of geometry for hair coloring tool support structure 520 may also be employed.

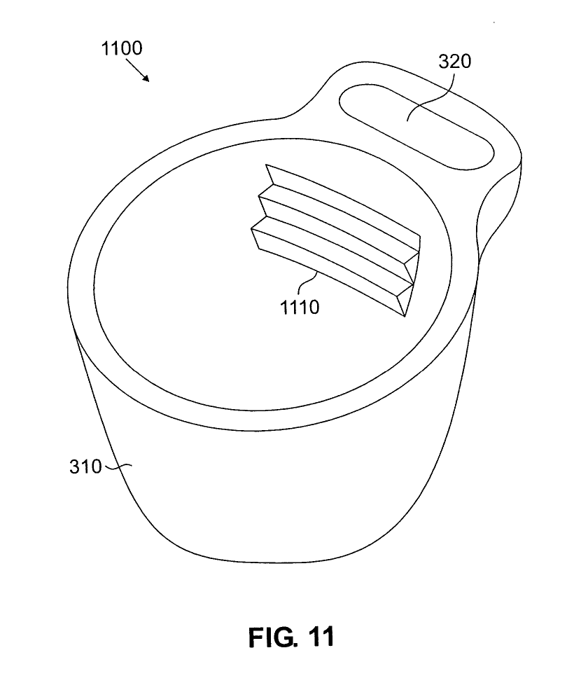

FIG. 11 illustrates a perspective top view of another advantageous embodiment of a hair coloring tool support stand 1100. Hair coloring tool support stand 1100 comprises a hair coloring tool support structure 1110 that does not extend down to the bottom of the hair coloring tool support stand 1100. Hair coloring tool support structure 1110 may be formed as an integral part of the wall of bowl 310 that is adjacent to receptacle 320. The hair coloring tool support structure 1110 may be used to remove excess hair coloring liquid from hair coloring tool 10 in the manner that has been previously described.

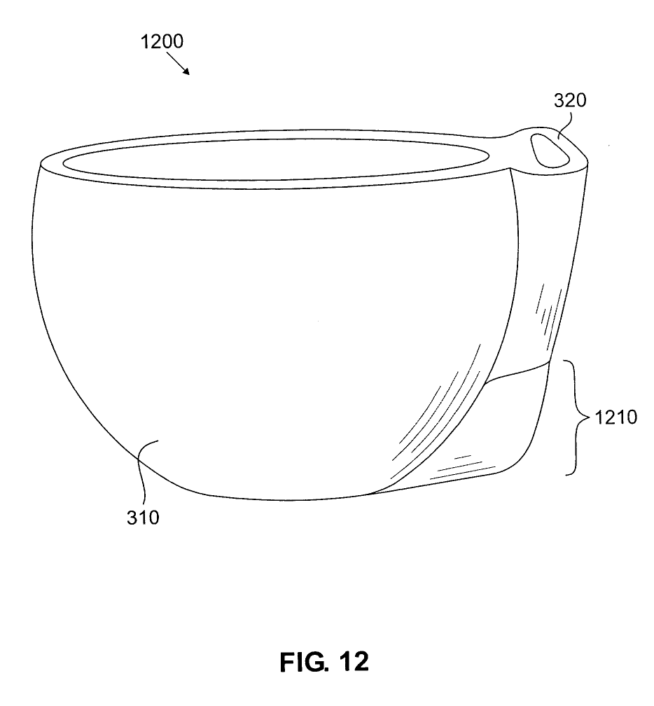

FIG. 12 illustrates a perspective side view of another advantageous embodiment a hair coloring tool support stand 1200. Hair coloring tool support stand 1200 comprises an extended foot portion 1210 that is located beneath the receptacle 320 of the hair coloring tool support stand 1200. The extended foot portion 1210 provides structural support for the bottom of receptacle 320. The extended foot portion 1210 also prevents the hair coloring support stand 1200 from tipping over in the direction of the receptacle 320.

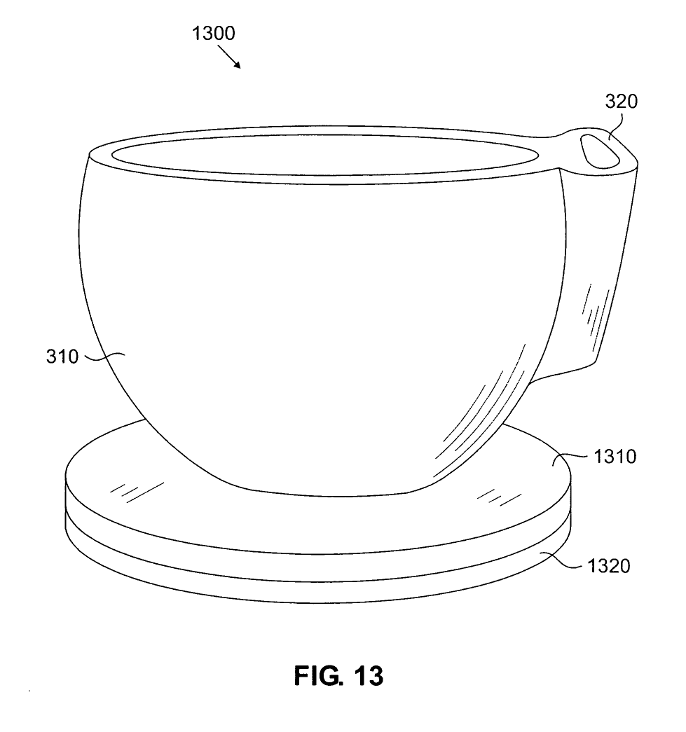

FIG. 13 illustrates a perspective side view of another advantageous embodiment a hair coloring tool support stand 1300. Hair coloring tool support stand 1300 comprises a base portion 1310 that is located beneath bowl 310 of the hair coloring support stand 1300. Base portion 1310 prevents the hair coloring tool support stand 1300 from tipping over in any direction. A layer of non-slip material 1320 (e.g., rubber) may be affixed to the bottom of base portion 1310 in order to prevent the base portion 1310 of hair coloring tool support stand 1300 from sliding when the hair coloring tool support stand 1300 is being used.

Although the present invention has been described using exemplary embodiments, various changes and modifications may be suggested to one skilled in the art. It is intended that the present invention encompass such changes and modifications as fall within the scope of the appended claims.

What is claimed is:

1. A hair coloring tool support stand comprising:
portions that form a bowl for containing a liquid; and
portions that form a receptacle for containing a hair coloring tool.

2. The hair coloring tool support stand as set forth in claim 1 wherein said portions that form said receptacle form a receptacle that has a width that is capable of receiving said hair coloring tool and a depth that is capable of holding said hair coloring tool when said hair coloring tool is placed within said receptacle.

3. The hair coloring tool support stand as set forth in claim 2 wherein said width of said receptacle is approximately two and five eighths inches and said depth of said receptacle is approximately one and two eighths inches.

4. The hair coloring tool support stand as set forth in claim 2 further comprising portions that form a foot portion of said receptacle that provides structural support to a bottom portion of said receptacle.

5. The hair coloring tool support stand as set forth in claim 1 further comprising:
a hair coloring tool support structure for supporting said hair coloring tool when said hair coloring tool is placed within said bowl of said hair coloring tool support stand.

6. The hair coloring tool support stand as set forth in claim 5 wherein said hair coloring tool support structure comprises a stair structure that comprises a plurality of stairs.

7. The hair coloring tool support stand as set forth in claim 5 wherein said plurality of stairs of said stair structure extend from a bottom of said bowl of said hair coloring tool support stand to a top of said bowl of said hair coloring tool support stand.

8. The hair coloring tool support stand as set forth in claim 7 wherein said plurality of stairs of said stair structure that extend from said bottom of said bowl to said top of said bowl are located adjacent to a wall of said bowl that is adjacent to said receptacle.

9. The hair coloring tool support stand as set forth in claim 7 wherein each of said plurality of stairs of said stair structure comprises a curved step having a center at a central axis of said bowl of said hair coloring tool support stand.

10. The hair coloring tool support stand as set forth in claim 7 wherein each of said plurality of stairs of said stair structure comprises a non curved step that is parallel to each of the other stairs in said plurality of stairs.

11. The hair coloring tool support stand as set forth in claim 7 wherein said stair structure comprises a circular sector having a center at a central axis of said bowl of said hair coloring support stand and having as boundaries radii from said central axis of said bowl to a wall of said bowl that is adjacent to said receptacle.

12. The hair coloring tool support stand as set forth in claim 1 wherein said portions that form said bowl form a bowl that has a top external diameter of approximately four inches and a top internal diameter of approximately three and five eighths inches.

13. The hair coloring tool support stand as set forth in claim 5 wherein said hair coloring tool support structure comprises a ladder structure that comprises a plurality of rungs.

14. The hair coloring tool support stand as set forth in claim 13 wherein said plurality of rungs of said ladder structure extend from a bottom of said bowl of said hair coloring tool support stand to a top of said bowl of said hair coloring tool support stand.

15. The hair coloring tool support stand as set forth in claim 14 wherein said plurality of rungs of said ladder structure that extend from said bottom of said bowl to said top of said bowl are located adjacent to a wall of said bowl that is adjacent to said receptacle.

16. A method of using a hair coloring tool support stand of the type that comprises a bowl for containing a liquid and a receptacle for containing a hair coloring tool, said method comprising the steps of:
placing said hair coloring tool within said bowl to contact a liquid within said bowl; and
placing said hair coloring tool within said receptacle when said hair coloring tool is not being used.

17. The method as set forth in claim 16 wherein said step of placing said hair coloring tool within said bowl to contact said liquid within said bowl comprises the step of:
placing said hair coloring tool on a hair coloring tool support structure within said bowl.

18. The method as set forth in claim 17 wherein said hair coloring tool support structure comprises one of: a stair structure and a ladder structure.

19. The method as set forth in claim 17 further comprising the step of:
removing excess liquid from said hair coloring tool by moving an application brush of said hair coloring tool against a portion of said hair coloring tool support structure within said bowl.

20. A hair coloring tool support stand comprising:
a bowl for containing a liquid; and
a receptacle formed within a wall of said bowl, wherein said receptacle is capable of holding a hair coloring tool when said hair coloring tool is placed within said receptacle.
